(19) United States
(12) Patent Application Publication
(10) Pub. No.: US 2018/0353630 A1
(43) Pub. Date: Dec. 13, 2018

(54) System and Methods for Tissue Sterilization

(71) Applicant: CritiTech, Inc., Lawrence, KS (US)

(72) Inventors: Joseph Farthing, Lawrence, KS (US); Michael Baltezor, Lawrence, KS (US)

(21) Appl. No.: 16/064,258

(22) PCT Filed: Dec. 23, 2016
(86) PCT No.: PCT/US16/68515

§ 371 Date: Jun. 20, 2018

Related U.S. Application Data
(60) Provisional application No. 62/387,412, filed on Dec. 24, 2015.
(60) Provisional application No. 62/287,137, filed on Jan. 26, 2016.

(51) Int. Cl. A61L 2/00

(52) U.S. Cl. CPC A61L 2/0088; A61L 2202/21; A61L 2202/15; A61L 2/0011

(57) ABSTRACT

Disclosed here are methods for tissue sterilization involving producing sonic energy from one or more sonic energy sources located at least partially within a pressurized chamber; and contacting a biological tissue positioned in the pressurized chamber with a supercritical fluid for a first time period effective to sterilize the biological tissue, where the contacting occurs exclusively at a temperature and pressure at or above the critical point of the supercritical fluid.

### CROSS REFERENCE

This application claims priority to U.S. Provisional Patent Application Nos. 62/387,412 filed Dec. 24, 2015 and 62/287,137 filed Jan. 26, 2016, each incorporated by reference herein in its entirety.

### BACKGROUND

Improved methods for sterilizing biological materials are needed in the tissue implantation or transplantation fields. Such methods need to provide consistent sterilization to levels necessary for tissue transplantation, maintain structural integrity of the tissue, and eliminate or significantly diminish residual sterilant and/or discoloration of the sterilized tissue.

### SUMMARY OF THE INVENTION

In one aspect, the invention provides tissue sterilization methods comprising

(a) producing sonic energy from one or more sonic energy sources located at least partially within a pressurized chamber; and

(b) contacting a biological tissue positioned in the pressurized chamber with a supercritical fluid for a first time period effective to sterilize the biological tissue, wherein the contacting occurs exclusively at a temperature and pressure at or above the critical point of the supercritical fluid.

In one embodiment, the method does not comprise creating an electric field or an electrostatic field within the pressurized chamber during the contacting. In another embodiment, the pressure is between about 1100 psi and about 1400 psi, such as between about 1150 psi and 1250 psi. In a further embodiment, the temperature is between about 31.1° C. and about 40° C. In another embodiment, the supercritical fluid is supercritical carbon dioxide. In a further embodiment, the wherein sonic energy source operates at a power of between about 60 watts to about 480 watts, such as between about 325 watts to about 445 watts. In another embodiment, the sonic energy source operates at a frequency of between about 10 kiloHertz (kHz) to about 40 kHz, such as about 20 kiloHertz. In a further embodiment, the sonic energy source comprises one of a sonic horn, a sonic probe, or a sonic plate. In another embodiment, the chamber is pressurized by injecting carbon dioxide into the chamber via an inlet of the chamber.

In one embodiment, the method further comprises removing the supercritical fluid from the pressurized chamber via an outlet of the pressurized chamber after the first time period. In another embodiment, there is no recirculation of the supercritical fluid to the pressurized chamber. In another embodiment, the first time period has a range between about 5 minutes to about 8 hours, such as between about 1 hour to about 2 hours. In a further embodiment, a flow rate of the supercritical fluid into the pressurized chamber during the first time period has a range from about 5 g/min to about 500 g/min, such as between about 50 g/min to about 100 g/min.

In another embodiment, the methods of the invention further comprise passing a fluid sterilant out of one or more nozzle orifices and into the pressurized chamber to produce an output stream of droplets of the fluid sterilant, wherein the nozzle orifice is positioned adjacent to the one or more sonic energy sources to thereby impart disruptive non-laminar flow of the fluid sterilant in the pressurized chamber; and contacting the biological tissue with the fluid sterilant for a second time period to further sterilize the biological tissue. In this embodiment, the method may further comprise removing the fluid sterilant from the pressurized chamber via the outlet after the second time period, such as removing the fluid sterilant to below organoleptic levels. In various embodiments, the fluid sterilant comprises a liquid selected from the group consisting of peroxides, epoxides, and/or carboxylic acid, including but not limited to hydrogen peroxide, acetic acid, and peracetic acid, and combinations thereof, and the fluid sterilant may be dissolved in water. In a specific embodiment, the fluid sterilant may comprise peracetic acid, such as peracetic acid having a concentration in the pressurized chamber in the range of about 0.001% to about 0.1%, such as between about 0.005% to about 0.015%. In various embodiments, the second time period has a range between about 2 hours to about 8 hours, such as between about 2 hours to about 3 hours. In another embodiment, the methods may further comprise, before the second time period, passing water out of the one or more nozzle orifices and into the pressurized chamber to produce an output stream of droplets of water. In a further embodiment, a flow rate of the fluid sterilant through each of the one or more nozzle orifices during the second time period has a range from about 0.5 mL/min to about 30 mL/min, such as between about 2 mL/min and about 4 mL/min. In one embodiment, each of the one or more nozzle orifices have a diameter in a range of about 20 μm to about 125 μm, such as between about 50 μm to about 70 μm.

In one embodiment of any of the embodiments of the methods of the invention, an inlet of the pressurized chamber has an inner diameter with a range from about 0.0625 inches to about 0.5 inches, such as between about 0.125 inches to about 0.25 inches. In another embodiment, the method excludes mechanical agitation within the pressurized chamber during the contacting. In a further embodiment, the methods further comprise depressurizing the chamber after the first and/or second time periods, wherein the depressurization occurs at a controlled rate.

### DESCRIPTION OF THE FIGURES

FIG. 1A illustrates a cross-section view of an example nozzle assembly, according to an example embodiment.

FIG. 1B illustrates a cross-section view of another example nozzle assembly, according to an example embodiment.

FIG. 2 is a block diagram of a method, according to an example embodiment.

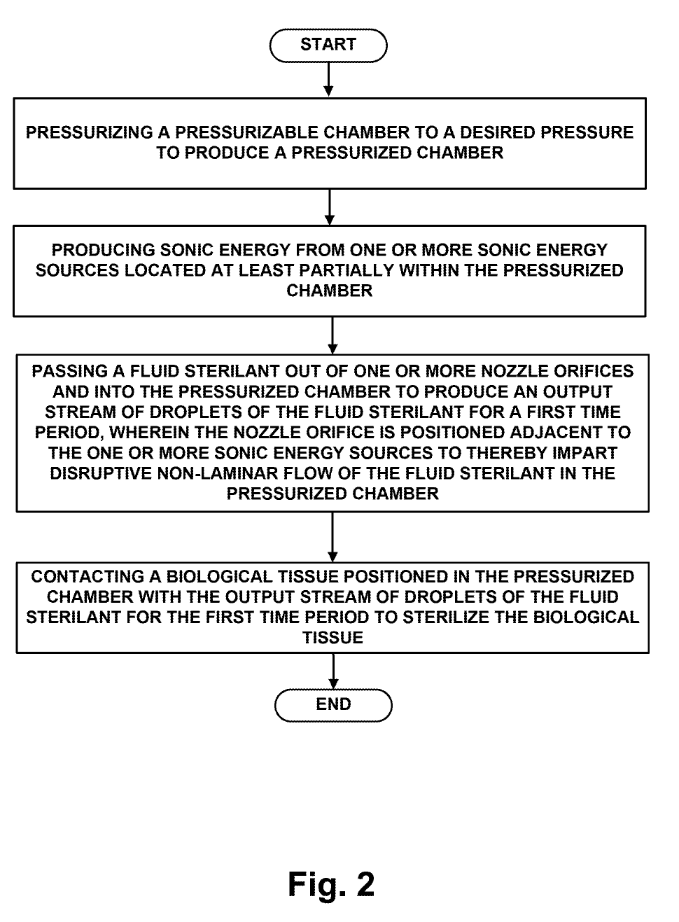

FIG. 3 is a block diagram of another method, according to an example embodiment.

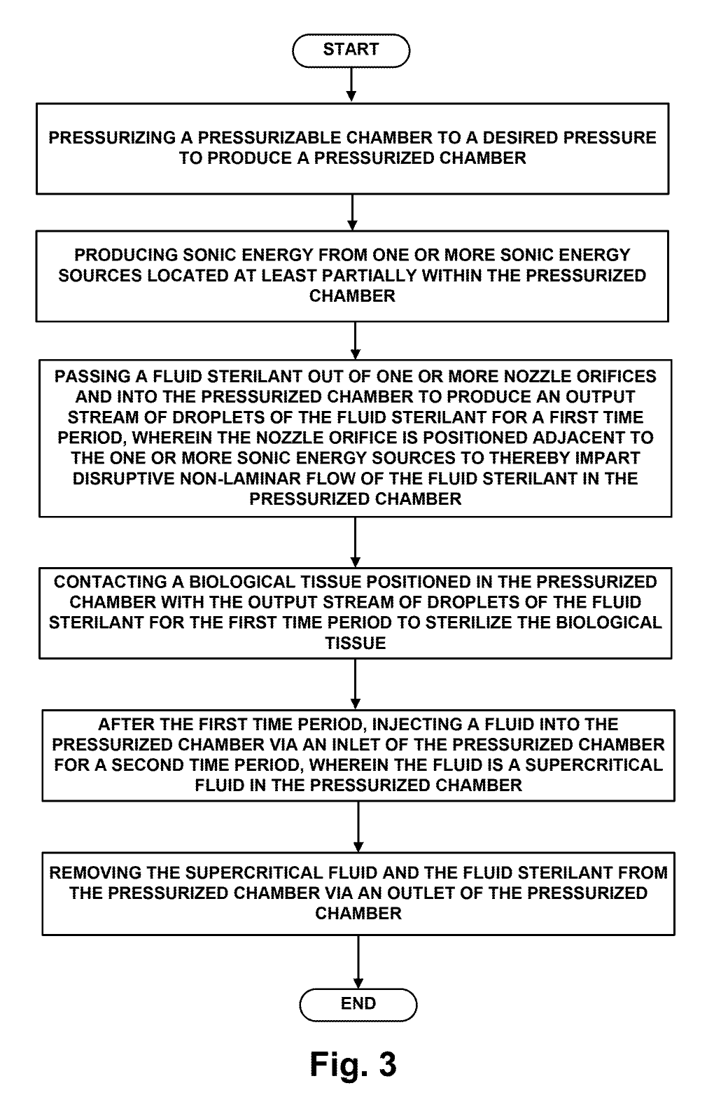

### DETAILED DESCRIPTION OF THE INVENTION

The present invention provides a method for sterilizing a biological tissue. The method may include (a) producing sonic energy from one or more sonic energy sources located at least partially within a pressurized chamber; and (b) contacting a biological tissue positioned in the pressurized chamber with a supercritical fluid for a first time period effective to sterilize the biological tissue, wherein the contacting occurs exclusively at a temperature and pressure above the critical point of the supercritical fluid.

The methods of the invention provide significant improvements to current sterilization techniques, in that they provide more consistent sterilization to levels necessary for tissue transplantation, maintain structural integrity of the tissue, and eliminate or significantly diminish residual sterilant and/or discoloration of the sterilized tissue.

In one embodiment, the method does not comprise creating an electric field or an electrostatic field within the pressurized chamber during the contacting. In one embodiment, the methods rely solely on the supercritical fluid for sterilization of the tissue (i.e.: no additional sterilant is added). In another embodiment, the methods further comprise passing a fluid sterilant out of one or more nozzle orifices and into the pressurized chamber to produce an output stream of droplets of the fluid sterilant, wherein the nozzle orifice is positioned adjacent to the one or more sonic energy sources to thereby impart disruptive non-laminar flow of the fluid sterilant in the pressurized chamber; and contacting the biological tissue with the fluid sterilant for a second time period to further sterilize the biological tissue.

The first and second time periods may be the same time period (i.e.: simultaneous), or may overlap (substantially or for a limited time period). In some embodiments, the method may further include removing the supercritical fluid and the fluid sterilant from the pressurized chamber via an outlet of the pressurized chamber.

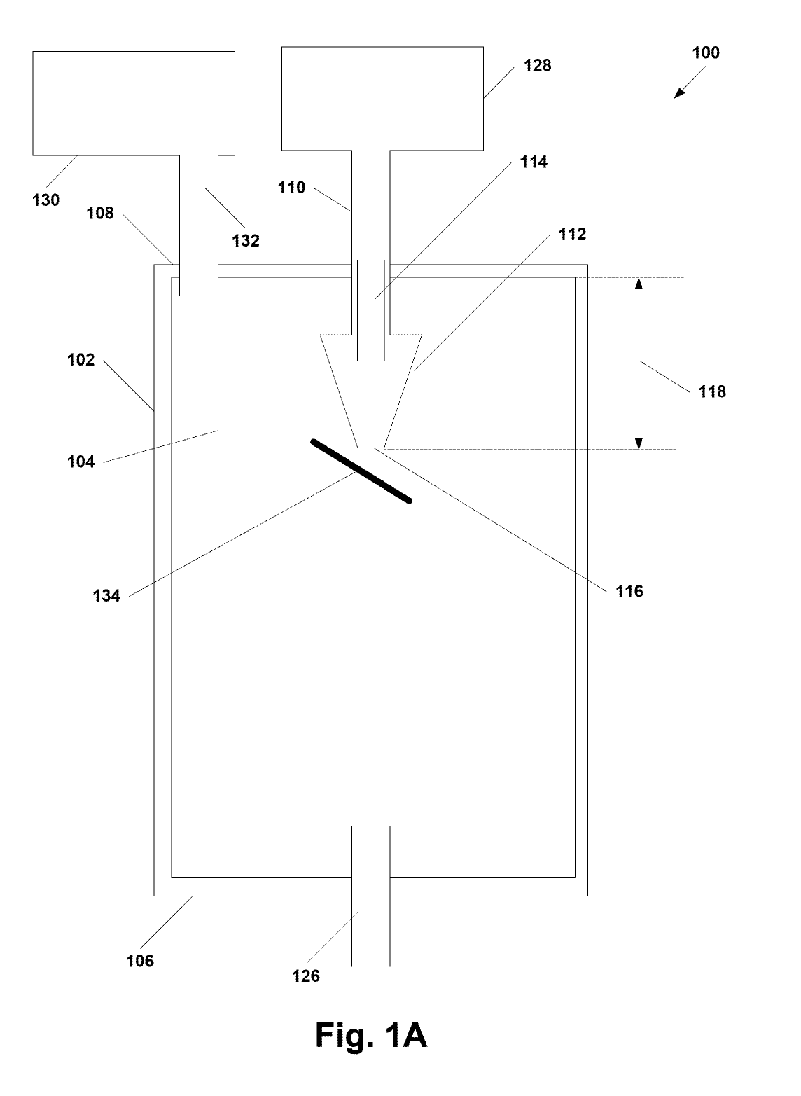

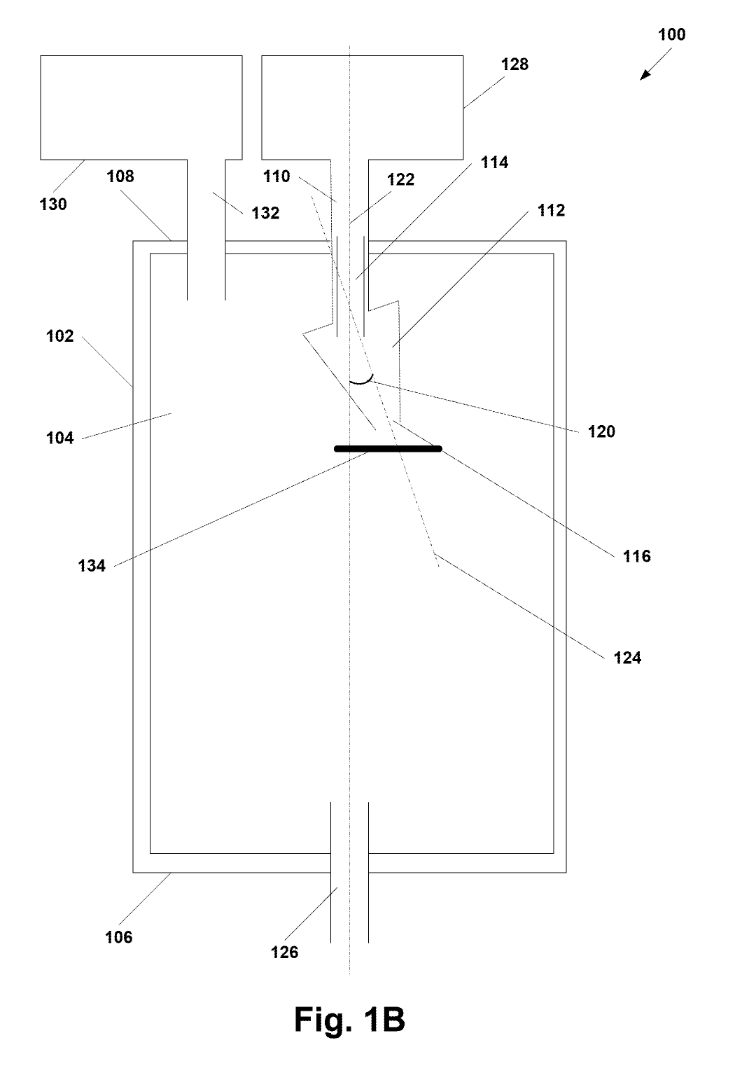

The pressurized chamber may be in fluid communication with a first reservoir and a second reservoir (when present). With reference to the Figures, as shown in FIG. 1A, the invention comprises a system 100 including a vessel 102 defining a pressurized chamber 104. The vessel 102 includes a distal end 106 and a proximal end 108. The system 100 further includes an inlet 110 of the pressurized chamber 104 at the proximal end 108 of the vessel 102. The system 100 further includes a nozzle 112 positioned within the pressurized chamber 104. As shown in FIG. 1A, the nozzle 112 includes an inlet tube 114 in fluid communication with the inlet 110 of the pressurized chamber 104. In addition, the nozzle 112 includes a nozzle orifice 116. Further, as shown in FIG. 1A, the nozzle 112 is adjustable to alter a distance 118 between the proximal end 108 of the vessel 102 and the nozzle orifice 116. As shown in FIG. 1B, the nozzle 112 is further adjustable to alter an angle 120 between a longitudinal axis of the vessel 122 and a longitudinal axis of the nozzle 124. In addition, the nozzle assembly 100 includes an outlet 126 of the pressurized chamber 104 at the distal end 106 of the vessel 102.

The pressurized chamber 104 may be in fluid communication with a first reservoir 128 and a second reservoir 130. The first reservoir 128 may include a supply of fluid, such as liquid carbon dioxide, that can be transformed into a supercritical fluid (for example, by pressurized above about 1070 psi and heated above about 31.1° C.), as described, for example, in U.S. Pat. Nos. 5,833,891 and 5,874,029, while the second reservoir 130 (when present) may include a supply of a fluid sterilant. Any suitable fluid that can be transformed into a supercritical fluid may be used in the methods of the invention; exemplary such fluids are disclosed in U.S. Pat. Nos. 5,833,891 and 5,874,029. In one non-limiting embodiment, such a fluid comprises liquid carbon dioxide. In one example, the nozzle orifice 116 may be in fluid communication with the second reservoir 130, while the first reservoir 128 is in fluid communication with the pressurized chamber 104 through an inlet 132 of the pressurized chamber 104.

The pressurized chamber 104 may be pressurized with a supply of a fluid, such as liquid carbon dioxide. The pressurized chamber 104 may be maintained at any pressure suitable for a given biological tissue, specific use, etc., so long as the pressurized chamber 104 is at or above the critical pressure for the supercritical fluid. In various embodiments, the pressurized chamber 104 may be maintained at a pressure between about 1100 psi and about 1400 psi, about 1100 psi and about 1300 psi, about 1100 psi and about 1250 psi, about 1100 psi and about 1200 psi, about 1150 psi and about 1400 psi, about 1150 psi and about 1300 psi, about 1150 psi and about 1250 psi, about 1200 psi and about 1400 psi, about 1200 psi and about 1350 psi, about 1200 psi and about 1300 psi, about 1250 psi and about 1400 psi, about 1250 psi and about 1350 psi, or about 1250 psi and about 1300 psi. In one example, the pressurized chamber 104 may be pressurized with liquid carbon dioxide.

Further, the pressurized chamber 104 may be maintained at any temperature suitable for a given biological tissue, specific use, etc., so long as the temperature of the pressurized chamber 104 is at or above the critical temperature for the a supercritical fluid. In various embodiments, the pressurized chamber 104 may be maintained at a temperature between about 31.1° C. and about 40° C., about 31.1° C. and about 38° C., about 31.1° C. and about 35° C., about 31.1° C. and about 33° C., about 33° C. and about 40° C., about 33° C. and about 38° C., about 33° C. and about 35° C., about 35° C. and about 40° C., about 35° C. and about 38° C., or about 35° C. and about 37° C.

Once the chamber is pressurized to a desired pressure, the one or more sonic energy sources may be turned on to produce sonic energy within the pressurized chamber. The one or more sonic energy sources may be positioned at least partially within the pressurized chamber. As described above and as shown in FIG. 1A, the nozzle 112 may be adjustable to alter a distance 118 between the proximal end 108 of the vessel 102 and the nozzle orifice 116 of the nozzle 112. In addition, as shown in FIG. 1B, the nozzle 112 may be adjustable to alter an angle 120 between a longitudinal axis of the vessel 122 and a longitudinal axis of the nozzle 124. In one example, both the angle of the nozzle 112 and the vertical position of the nozzle 112 may be adjusted manually by a user. For example, the nozzle 112 may be positioned on a vertical support that can be adjusted to alter the distance 118 between the proximal end 108 of the vessel 102 and the nozzle orifice 116 of the nozzle 112. Further, the nozzle 112 may be rotated manually to adjust the angle 120 between the longitudinal axis of the vessel 122 and the longitudinal axis of the nozzle 124.

In another example, the system 100 may include a motor coupled to the nozzle 112. In various examples, the motor may be configured to alter the distance 118 between the proximal end 108 of the vessel 102 and the nozzle orifice 116 of the nozzle 112 and/or alter the angle 120 between the longitudinal axis of the vessel 122 and the longitudinal axis of the nozzle 124. Such a motor may be an electric motor powered by electrical power, or may be powered by a number of different energy sources, such as a gas-based fuel or solar power. The motor may be coupled directly or indirectly to the nozzle 112, such that when the motor is turned on the distance 118 between the proximal end 108 of the vessel 102 and the nozzle orifice 116 of the nozzle 112 increases or decreases depending on the direction the motor rotates. The motor may be coupled to a series of gears that adjusts the distance 118 between the proximal end 108 of the vessel 102 and the nozzle orifice 116 of the nozzle 112 and/or adjusts the angle 120 between the longitudinal axis of the vessel 122 and the longitudinal axis of the nozzle 124, or the motor may be coupled to a pulley system that adjusts the distance 118 between the proximal end 108 of the vessel 102 and the nozzle orifice 116 of the nozzle 112 and/or adjusts the angle 120 between the longitudinal axis of the vessel 122 and the longitudinal axis of the nozzle 124. Other configurations are possible as well.

In another example, the nozzle 112 assembly may include an actuator coupled to the nozzle 112, where the actuator alters the distance 118 between the proximal end 108 of the vessel 120 and the nozzle orifice 116 of the nozzle 112 and/or alters the angle 120 between the longitudinal axis of the vessel 122 and the longitudinal axis of the nozzle 124. Such an actuator may be an electro-mechanical actuator, including an electric motor that converts a rotary motion of the electric motor to a linear displacement via a linkage system. Other potential actuators are possible as well, such as hydraulic actuators, pneumatic actuators, piezoelectric actuators, linear motors, or telescoping linear actuators, as examples.

In further examples, the system 100 may include a plurality of nozzles, with each nozzle positioned at a different angle between a longitudinal axis of the vessel 122 and a longitudinal axis of the nozzle 124 and/or a different distance between the nozzle orifice 116 and the sonic energy source 134. Other example embodiments are possible as well.

In one example, as shown in FIGS. 1A and 1B, the system further includes a sonic energy source 134 positioned adjacent to the nozzle orifice 116 of the nozzle 112. Any suitable source of sonic energy may be used that is compatible with the methods of the invention, including but not limited to sonic horn, a sonic probe, or a sonic plate. In one example, the sonic energy source 134 may include a sonic probe extending at least partially within the pressurized chamber 104. In another example, the sonic energy source 134 may include a sonic surface positioned in the pressurized chamber 104. The sonic waves from the sonic energy source 134 cause the sterilant to penetrate the biologic tissue. In one example, 134 is positioned at an angle of 45 degrees with respect to the longitudinal axis of the nozzle 124. Other angles are possible as well. In one example, the sonic energy source 134 may be adjustable to alter a distance between the nozzle orifice 116 of the nozzle 112 and the sonic energy source 134. Further, the sonic energy source 134 may be adjustable to alter an angle between the sonic energy source 134 and the longitudinal axis of the nozzle 124. The sonic energy source 134 may produce sonic energy with any amplitude suitable for a given biological tissue, specific use, etc. In various further embodiments, the sonic energy source 134 may produce sonic energy using any power suitable for a given biological tissue, specific use, etc.

In various embodiments, the sonic energy source 134 operates at a power between about 60 watts and about 480 watts, about 60 watts and about 450 watts, about 60 watts and about 400 watts, about 60 watts and about 350 watts, about 60 watts and about 300 watts, about 60 watts and about 250 watts, about 60 watts and about 200 watts, about 60 watts and about 150 watts, about 60 watts and about 100 watts, about 100 watts and about 480 watts, out 100 watts and about 450 watts, out 100 watts and about 400 watts, out 100 watts and about 350 watts, out 100 watts and about 300 watts, out 100 watts and about 250 watts, out 100 watts and about 200 watts, about 200 watts and about 450 watts, about 200 watts and about 400 watts, about 200 watts and about 350 watts, about 200 watts and about 300 watts, about 200 watts and about 250 watts, about 280 watts and about 450 watts, about 280 watts and about 400 watts, about 280 watts and about 360 watts, about 280 watts and about 300 watts, or about 325 watts to about 445 watts. In another embodiment, a frequency of between about 10 kiloHertz (kHz) to about 40 kHz, such as about 20 kHz is utilized. The frequency may be constant or variable.

The nozzle orifice 116 of the nozzle 112 may include a plurality of ridges to create a vortex within the nozzle 112 such that the solvent exits the nozzle 112 via turbulent flow. In another example, the nozzle 112 may include a porous fit interior to the nozzle 112 such that the solvent exits the nozzle 112 via turbulent flow. In yet another example, the nozzle orifice 116 of the nozzle 112 may have a small diameter (as discussed in additional detail below) such that the solvent exits the nozzle 112 via turbulent flow. These various embodiments that cause turbulent flow may assist in mixing the solvent with the anti-solvent within the pressurized chamber 104. The nozzle orifice 116 of the nozzle 112 may be located any suitable distance from the sonic energy source 134 for a given tissue and use.

In various further embodiments, the sonic energy source 134 produces sonic energy with an amplitude between about 1% and about 100% of the total power that can be generated using the sonic energy source. In light of the teachings herein, one of skill in the art can determine an appropriate sonic energy source having a specific total power output to be used. In one embodiment, the sonic energy source has a total power output of between about 60 watts to about 480 watts, such as between about 325 watts to about 445 watts. In light of the teachings herein, one of skill in the art can determine an appropriate frequency to be utilized on the sonic energy source 134. In one embodiment, a frequency of between about 18 and about 22 kHz on the sonic energy source is utilized. In various other embodiments, a frequency of between about 19 and about 21 kHz, about 19.5 and about 20.5, or, a frequency of about 20 kHz on the sonic energy source is utilized. Any suitable source of sonic energy may be used that is compatible with the methods of the invention, including but not limited to sonic horn, a sonic probe, or a sonic plate.

A potentially supercritical fluid (or liquid/gas form of the supercritical fluid) is injected into the pressurized chamber 104 via an inlet 110 of the pressurized chamber 104 for the first time period. Such a fluid (or gas) may be liquid carbon dioxide, for example, which transforms into supercritical carbon dioxide in the pressurized chamber 104. Further, the supercritical fluid may be removed from the pressurized chamber 104 via an outlet 126 of the pressurized chamber, thereby flushing the pressurized chamber 104 with the supercritical fluid for the first time period to remove excess fluid sterilant from the biological tissue. In one example, there is no recirculation of the supercritical fluid to the pressurized chamber 104, such that fresh fluid is supplied to and removed from the pressurized chamber 104 for the second time period. The one or more sonic energy sources 134 may continue to produce sonic energy in the pressurized chamber 104 during this flushing step.

The inlet of the pressurized chamber 110 may be positioned adjacent to the one or more sonic energy sources 134 to thereby impart disruptive non-laminar flow of the fluid in the pressurized chamber 104. As the fluid passes into the pressurized chamber 104, the fluid turns into a supercritical fluid and the supercritical fluid contacts the biological tissue positioned in the pressurized chamber 104 to sterilize the biological tissue.

The inlet 110 of the pressurized chamber 104 may have any suitable inner diameter, including but not limited to a range from about 0.0625 inches to about 0.5 inches, such as between about 0.125 inches and about 0.25 inches.

During the pressurization step, the supercritical fluid may be passed into the pressurized chamber 104 through the inlet 110 for any period of time suitable for a given biological tissue, specific use, etc. In various embodiments, the supercritical fluid is passed into the pressurized chamber 104 through the inlet 110 for a period of between about 5 minutes to about 8 hours, about 5 minutes to about 7 hours, about 5 minutes to about 6 hours, about 5 minutes to about 5 hours, about 5 minutes to about 4 hours, about 5 minutes to about 3 hours, about 5 minutes to about 2 hours, about 5 minutes to about 1 hour, about 30 minutes to about 8 hours, about 30 minutes to about 7 hours, about 30 minutes to about 6 hours, about 30 minutes to about 5 hours, about 30 minutes to about 4 hours, about 30 minutes to about 3 hours, about 30 minutes to about 2 hours, about 30 minutes to about 1 hour, about 1 hour to about 7 hours, about 1 hour to about 6 hours, about 1 hour to about 5 hours, about 1 hour to about 4 hours, about 1 hour to about 3 hours, about 1 hour to about 2 hours, about 1 hour, about 2 hours, about 3 hours, about 4 hours, about 5 hours, about 6 hours, about 7 hours, or about 8 hours.

The flow rate of the fluid into the pressurized chamber 104 during the first time period can be adjusted as high as possible to optimize output but below the pressure limitations for the equipment, including the inlet 110 of the pressurized chamber 104. As such, the flow rate of the fluid may be any suitable flow rate for a given biological tissue, specific use, etc. In various embodiments, a flow rate of the fluid into the pressurized chamber 104 during the first time period has a range from about 5 grams per minute (g/min) to about 500 g/min, about 5 g/min to about 450 g/min, about 5 g/min to about 400 g/min, about 5 g/min to about 350 g/min, about 5 pgm to about 300 g/min, about 5 g/min to about 250 g/min, about 5 g/min to about 200 g/min, about 5 g/min to about 150 g/min, about 5 g/min to about 100 g/min, about 5 g/min to about 50 g/min or about 5 g/min to about 25 g/min.

The method may further comprise passing a fluid sterilant out of one or more nozzle orifices 116 and into the pressurized chamber 104 to produce an output stream of droplets of the fluid sterilant. The one or more nozzle orifices 116 may be positioned within the pressurized chamber 104. Further, the one or more nozzle orifices 116 may be positioned adjacent to the one or more sonic energy sources 134 to thereby impart disruptive non-laminar flow of the fluid sterilant in the pressurized chamber 104. In particular, the sonic waves from the one or more sonic energy sources 134 causes intermolecular forces of the liquid sterilant to break, thereby imparting disruptive non-laminar flow of the fluid sterilant to provide better coverage of the liquid sterilant on and in the biological tissue. The sonic energy may uniformly combine the sterilant with the carrier supercritical fluid, either as a suspension of droplets or in a fully dissolved state. In this manner, the sonic energy causes rapid and uniform distribution of the sterilant throughout the vessel. Thus the biological tissue to be sterilized is exposed to a uniform dose of sterilant throughout the vessel, which in turn is needed for uniform sterilization. Lack of such disruptive non-laminar flow may result in non-uniform sterilant concentration that in turn would produce non-uniform sterilization and require longer times to achieve complete sterilization of the biological tissue.

The fluid sterilant may be any fluid suitable to sterilize a given biological tissue, or suitable for any specific use, etc. In various embodiments, the fluid sterilant may include a liquid selected from the group consisting of peroxides, carboxylic acid, including but not limited to hydrogen peroxide, acetic acid, and peracetic acid, and combinations thereof. In one particular example, the fluid sterilant comprises peracetic acid having a concentration suitable for a given biological tissue, specific use, etc. In various embodiments, the concentration of peracetic acid in the supercritical carbon dioxide in the pressurized chamber 104 may be between about 0.001% and about 0.1%, about 0.001% and about 0.075%, about 0.001% and about 0.05%, about 0.001% and about 0.025%, about 0.001% and about 0.015%, about 0.002% and about 0.1%, about 0.002% and about 0.075%, about 0.002% and about 0.05%, about 0.002% and about 0.025%, about 0.002% and about 0.015%, about 0.005% and about and about 0.1%, about 0.005% and about 0.075%, about 0.005% and about 0.05%, about 0.005% and about 0.025%, or about 0.005% and about 0.015%.

In one example embodiment, the fluid sterilant is dissolved in water. In such an example, the water may be combined with the peracetic acid (or other sterilant) to achieve a desired concentration, and subsequently passed through the one or more nozzle orifices. In another example, the peracetic acid (or other sterilant) may be passed out of a first nozzle orifice and into the pressurized chamber, while water may be passed out of a second nozzle orifice and into the pressurized chamber. In yet another example, the water is passed through the one or more nozzle orifices at a first point in time, and then the fluid sterilant is passed through the same the one or more nozzle orifices at a second point in time to produce an output stream of droplets of water. In such an example, water may be passed through the one or more nozzle orifices for any period of time suitable to sterilize a given biological tissue, specific use, etc. In various embodiments, the water may be passed out of the one or more nozzle orifices 116 and into the pressurized chamber 104 for a period of between about 30 seconds to about 1 hour, about 30 seconds to about 45 minutes, about 30 seconds to about 30 minutes, about 30 seconds to about 15 minutes, about 30 seconds to about 10 minutes, about 30 seconds to about 5 minutes, about 30 seconds to about 2 minutes, about 1 hour, about 45 minutes, about 30 minutes, about 15 minutes, about 10 minutes, about 5 minutes, about 2 minutes, about 1 minute, or about 30 seconds. During the sterilizing step, the fluid sterilant and water may be maintained in the pressurized chamber as a closed system.

The one or more nozzle orifices 116 may be configured to create a vortex within each such nozzle such that the sterilant exits a given nozzle via turbulent flow. Such an embodiment may assist in providing improved coverage of the sterilant on the biological tissue in the pressurized chamber.

In operation, the fluid sterilant may be passed out of the one or more nozzle orifices 116 and into the pressurized chamber for any period of time suitable to sterilize a given biological tissue, specific use, etc. In various embodiments, the fluid sterilant is passed out of the one or more nozzle orifices and into the pressurized chamber for a period of between about 2 hours to about 8 hours, about 2 hours to about 7 hours, about 2 hours to about 6 hours, about 2 hours to about 5 hours, about 2 hours to about 4 hours, about 2 hours to about 3 hours, about 2 hours, about 3 hours, about 4 hours, about 5 hours, about 6 hours, about 7 hours, or about 8 hours.

Each of the one or more nozzle orifices 116 may have any suitable diameter that is suitable for a given biological tissue, specific use, etc. In other various embodiments, each of the one or more nozzle orifices 116 have a diameter of between about 20 μm and about 125 μm, about 20 μm and about 115 μm, about 20 μm and about 100 μm, about 20 μm and about 90 μm, about 20 μm and about 80 μm, about 20 μm and about 70 μm, about 20 μm and about 60 μm, about 20 μm and about 50 μm, about 20 μm and about 40 μm, about 20 μm and about 30 μm, between about 30 μm and about 125 μm, about 30 μm and about 115 μm, about 30 μm and about 100 μm, about 30 μm and about 90 μm, about 30 μm and about 80 μm, about 30 μm and about 70 μm, about 30 μm and about 60 μm, about 30 μm and about 50 μm, about 30 μm and about 40 μm, between about 40 μm and about 125 μm, about 40 μm and about 115 μm, about 40 μm and about 100 μm, about 40 μm and about 90 μm, about 40 μm and about 80 μm, about 40 μm and about 70 μm, about 40 μm and about 60 μm, about 40 μm and about 50 μm, between about 50 μm and about 125 μm, about 50 μm and about 115 μm, about 50 μm and about 100 μm, about 50 μm and about 90 μm, about 50 μm and about 80 μm, about 50 μm and about 70 μm, about 50 μm and about 60 μm, between about 60 μm and about 125 μm, about 60 μm and about 115 μm, about 60 μm and about 100 μm, about 60 μm and about 90 μm, about 60 μm and about 80 μm, about 60 μm and about 70 μm, between about 70 μm and about 125 μm, about 70 μm and about 115 μm, about 70 μm and about 100 μm, about 70 μm and about 90 μm, about 70 μm and about 80 μm, between about 80 μm and about 125 pin, about 80 μm and about 115 μm, about 80 μm and about 100 μm, about 80 μm and about 90 μm, between about 90 μm and about 125 μm, about 90 μm and about 115 μm, about 90 μm and about 100 μm, between about 100 μm and about 125 μm, about 100 μm and about 115 μm, between about 115 μm and about 125 μm, about 20 μm, 30 μm, 40 μm, 50 μm, 60 μm, 70 μm, 80 μm, 90 μm, 100 μm, 115 μm, or about 120 μm.

In further examples, the system 100 may include a plurality of nozzles, with each nozzle positioned at a different angle between a longitudinal axis of the pressurized chamber 122 and a longitudinal axis of the nozzle 124 and/or a different distance between the nozzle orifice 116 and the sonic energy source 134. In such an example, each of the plurality of nozzles may be in fluid communication with the first reservoir 128 containing the sterilant. In another example, each of the plurality of nozzles may be in fluid communication with a different reservoir, where each reservoir includes a different sterilant. In such an example, a given nozzle of the plurality of nozzles may be chosen for a given sterilization process for a particular biological tissue, for example.

Each of the one or more nozzle orifices 116 may be spaced away from the one or more sonic energy sources 134 at any suitable distance for a given tissue and use.

The flow rate of the sterilant can be adjusted as high as possible to optimize output but below the pressure limitations for the equipment, including the nozzle orifice 116. As such, the flow rate of the sterliant may be any suitable flow rate for a given biological tissue, specific use, etc. In various embodiments, a flow rate of the sterilant through the nozzle orifice 116 has a range from about 0.5 mL/min to about 30 mL/min. In various further embodiments, the flow rate is between about 0.5 mL/min to about 25 mL/min, 0.5 mL/min to about 20 mL/min, 0.5 mL/min to about 15 mL/min, 0.5 mL/min to about 10 mL/min, about 1 mL/min to about 30 mL/min, about 1 mL/min to about 25 mL/min, about 1 mL/min to about 20 mL/min, 1 mL/min to about 15 mL/min, about 1 mL/min to about 10 mL/min, about 2 mL/min to about 30 mL/min, about 2 mL/min to about 25 mL/min, about 2 mL/min to about 20 mL/min, about 2 mL/min to about 15 mL/min, about 2 mL/min to about 10 mL/min, about 2 mL/min to about 8 mL/min, or about 2 mL/min to about 4 mL/min.

As discussed above, after the fluid sterilant has been passed out of the one or more nozzle orifices 116 and into the pressurized chamber 104 for the time period described above, the method may further include refraining the fluid sterilant from passing out of the one or more nozzle orifices 116.

In a further embodiment of any of the embodiments or combinations of embodiments disclosed herein, the methods exclude mechanical agitation within the pressurized chamber during the contacting. In a further embodiment of any of the embodiments or combinations of embodiments disclosed herein, the depressurizing comprises depressurizing the chamber after the first and/or second time period at a controlled rate

In addition, for the method and other processes and methods disclosed herein, the method steps may represent functionality and operation of one possible implementation of present embodiments. In this regard, each method step may represent a module, a segment, or a portion of program code, which includes one or more instructions executable by a processor or computing device for implementing specific logical functions or steps in the process. The program code may be stored on any type of computer readable medium, for example, such as a storage device including a disk or hard drive. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Since many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

### Examples

A non-limiting, exemplary protocol for that has been used multiple times and resulted in sterilization of greater than 10⁶ with no tissue discoloration of Achilles and Hemi bone-tendon interfaces, both with and without sterilant (no residual peracetic acid, when used as sterilant):

1. Tissue samples were loaded into the chamber, using plastic wire ties to attach samples to tray.
2. The chamber was closed and pressurized to 1250 psi.
3. After pressurization was achieved, the chamber heaters were activated to warm the chamber to 38° C.; and they were maintained between 35° C. and 40° C. during the entire process.
4. Once the desired temperature was reached the sonicators were activated to 385 watts. Three Q-sonica sonic horns were used, spaced evenly along the top of the horizontally oriented chamber. The sonic probes, were inserted so that the tips were even with the interior surface of the chamber.
5. After the sonicators were activated and the temperature was stable, 5.00 mL of sterile water (optional) was sprayed into the chamber at a flowrate of 2 mL/min.
6. After completion of the water spray, 2.5 mL of 39% peracetic acid (PAA) (optional) may be sprayed into the chamber at a flowrate of 2 mL/min. Note: We have been successful at sterilizing Achilles/bone without PAA; instead water was sprayed water at this step (optional).
7. The process from post-pressurization (i.e.: after step 2) was allowed to continue for 120 min with sonication remaining on for the entire time period, adjusting keeping the temperature between 35°−40° C.
8. After completion of the process, clean CO2 was flushed through the chamber/system for 15 min to remove the peracetic acid (when PAA was used).
9. The sonicator was then stopped, and the heaters were stopped.
10. The system was then depressurized in a constant and controlled manner, at 100 psi/min. At this rate, the depressurization process was complete in approximately 12.5 min
11. The samples were removed.

1. A sterilization method comprising:
(a) producing sonic energy from one or more sonic energy sources located at least partially within a pressurized chamber; and
(b) contacting a biological tissue positioned in the pressurized chamber with a supercritical fluid for a first time period effective to sterilize the biological tissue, wherein the contacting occurs exclusively at a temperature and pressure at or above the critical point of the supercritical fluid.

2. The method of claim 1, wherein the method does not comprise creating an electric field or an electrostatic field within the pressurized chamber during the contacting.

3. The method of claim 1, wherein the pressure is between about 1100 psi and about 1400 psi.

4. The method of claim 1, wherein the temperature is between about 31.1° C. and about 40° C.

5. The method of claim 1, wherein the supercritical fluid is supercritical carbon dioxide.

6. The method of claim 1, wherein the sonic energy source operates at a power of between about 60 watts to about 480 watts.

7. The method of claim 1, wherein sonic energy source operates at a frequency of between about 10 kiloHertz (kHz) to about 40 kHz.

8. The method of claim 1, wherein the sonic energy source comprises one of a sonic horn, a sonic probe, or a sonic plate.

9. The method of claim 1, wherein the chamber is pressurized by injecting carbon dioxide into the chamber via an inlet of the chamber.

10. The method of claim 1, further comprising removing the supercritical fluid from the pressurized chamber via an outlet of the pressurized chamber after the first time period.

11. The method of claim 1, wherein there is no recirculation of the supercritical fluid to the pressurized chamber.

12.-13. (canceled)

14. The method of claim 1, further comprising:
passing a fluid sterilant out of one or more nozzle orifices and into the pressurized chamber to produce an output stream of droplets of the fluid sterilant, wherein the nozzle orifice is positioned adjacent to the one or more sonic energy sources to thereby impart disruptive non-laminar flow of the fluid sterilant in the pressurized chamber; and
contacting the biological tissue with the fluid sterilant for a second time period to further sterilize the biological tissue.

15. The method of claim 14, further comprising removing the fluid sterilant from the pressurized chamber via the outlet after the second time period.

16. The method of claim 14, wherein the fluid sterilant comprises a liquid selected from the group consisting of peroxides, epoxides, and/or carboxylic acid, including but not limited to hydrogen peroxide, acetic acid, and peracetic acid, and combinations thereof.

17.-19. (canceled)

20. The method of claim 14, wherein the second time period has a range between about 2 hours to about 8 hours.

21. The method of claim 14, further comprising:
before the second time period, passing water out of the one or more nozzle orifices and into the pressurized chamber to produce an output stream of droplets of water.

22. (canceled)

23. The method of claim 1, wherein an inlet of the pressurized chamber has an inner diameter with a range from about 0.0625 inches to about 0.5 inches.

24. The method of claim 14, wherein each of the one or more nozzle orifices have a diameter in a range of about 20 μm to about 125 μm.

25. The method of claim 1, wherein the method excludes mechanical agitation within the pressurized chamber during the contacting.

26. The method of claim 1, further comprising depressurizing the chamber after the first and/or second time periods, wherein the depressurization occurs at a controlled rate.
